(19) United States
(12) Patent Application Publication
(10) Pub. No.: US 2004/0191176 A1
(43) Pub. Date: Sep. 30, 2004

(54) Formulations for treatment of pulmonary disorders

(76) Inventor: Leonard W. Kaplan, Brookline, MA (US)

Correspondence Address: REED & EBERLE LLP, 800 MENLO AVENUE, SUITE 210, MENLO PARK, CA, 94025, US

(21) Appl. No.: 10/402,771

(22) Filed: Mar. 28, 2003

(51) Int. Cl. A61L 9/04; A61K 31/137

(52) U.S. Cl. 424/45; 514/649

(57) ABSTRACT

Pharmaceutical formulations useful for the treatment of pulmonary disorders are described. These compositions are formulated for pulmonary administration and contain a bronchodilator, a vasoconstrictor, a corticosteroid, and an optional pharmaceutically acceptable carrier. Drug delivery devices and dosage forms for housing and/or dispensing the formulations are also described.

### TECHNICAL FIELD

This invention relates to pharmaceutical formulations useful for the treatment of pulmonary disorders such as chronic obstructive pulmonary disease and asthma. More particularly, the invention relates to formulations containing a bronchodilator, a vasoconstrictor, a corticosteroid and an optional carrier.

### BACKGROUND

Asthma is a chronic episodic inflammatory disease of the airways. A widespread narrowing of the air passages results from a combination of constriction of the muscles that encircle the bronchioles, inflammation of the bronchial mucosa, and secretion of inspissated or thick tenacious mucous. Affecting up to 5% of the population, asthma is manifested by coughing, wheezing and dyspnea or shortness of breath.

Asthma is an episodic and variable disorder in that there are acute exacerbations of symptoms, interspersed with periods in which the patient is asymptomatic. The intensity of symptoms can vary from one occurrence to another and both the intermediate and late phases of this disorder can be both erratic and unpredictable. During the early phase, mediators such as histamine, bradykinin, leukotrienes, platelet activating factor, and prostaglandins, cause bronchoconstriction by contraction of smooth muscles, increased mucous secretion and mucosal edema. During the late phase, infiltrating cells such as eosinophils, macrophages, and platelets, enhance the airway narrowing.

Typical treatment regimens involve administering several different types of bronchodilators by inhaled or oral dosage forms. These include theophylline and alpha and beta adrenergic agonists. Since patients experiencing an asthmatic attack often require immediate relief to prevent further complications, e.g., hypoxia and even death, it is critical that the bronchodilator exerts its pharmacological actions as quickly as possible. Therefore, inhalation therapy with a bronchodilator is particularly preferred as it provides for a relatively fast onset of action by placing the drug in direct contact with the target tissues, i.e., pulmonary tissues. Consequently, bronchodilators are generally administered via a metered-dose inhaler for oral inhalation, although other vehicles, e.g., tablets and syrups, are also available.

Bronchodilator therapy, however, is not without drawbacks. For example, bronchodilators relieve only the immediate symptoms of asthma, e.g., shortness of breath and difficulty breathing. Thus, monotherapy with a bronchodilator will not decrease the number of asthmatic episodes a predisposed individual will experience. As the pathophysiology of asthma became better understood, it became apparent that that the disease not only involved a bronchospastic process but also an inflammatory one. Treatment was then modified to include mast cell inhibitors such as sodium cromolyn in conjunction with β₂ agonist bronchodilators.

Other therapeutic regimes involved inhaled corticosteroids as an attempt to prevent the onset of wheezing and shortness of breath, as well as minimize the need for oral steroids. Steroids such as beclomethasone, triamcinolone, budesonide and fluticasone were then added to the list of agents actively prescribed to patients suffering from asthma. Examples of these combinations include: β₂ agonists in combination with glucocorticosteroids, U.S. Pat. No. 6,030,604 to Trofast; bronchodilators in combination with beclomethasone dipropionate monohydrate, U.S. Pat. No. 5,688,782 to Neale et al.; salmeterol in combination with fluticasone, Chapman et al. (1999) Can. Respir. J. 6(1):45-51; and salmeterol in combination with beclomethasone, Kelson et al. (1999) J. of Asthma 36(8):703-715. Since inflammation can often trigger an asthma attack, administration of an antiinflammatory corticosteroid was found to reduce the number of asthmatic attacks an individual may experience. Thus, the combination of a bronchodilator and corticosteroid not only provided immediate relief of asthmatic symptoms, but reduced the likelihood of a subsequent asthmatic attack as well.

Unfortunately, as these inhaled steroids need to be taken regularly as a preventive measure, patient compliance was poor because of the slow onset of action of this type of medication. Patients often forgot to use their inhaler, or claimed that they did not feel better after taking this type of medication, or did not take the medicine until after the symptoms started at which point the preventative benefits of the medication were lost. Pharmaceutical companies attempted to improve compliance by developing stronger steroids that required fewer inhaled doses to deliver the required therapeutically effective amount. However, this still did not improve patient compliance since even the stronger steroids could still not provide an immediate onset of relief.

Longer acting-bronchodilators such as salmeterol were also developed in conjunction with orally inhaled steroids. However, such products did not take into consideration the episodic nature of asthmatic attacks in that the attacks can last anywhere from a few minutes up to a few months, or even longer. Longer acting bronchodilators still took 30-40 minutes to provide relief so patients were often required to have two separate inhalers, one for a short acting beta-2 agonist and one for the salmeterol/steroid combination.

Another issue that has not been addressed by current drug regimens for the treatment of asthma is the mucosal edema that occurs as a result of the inflammation of the bronchial mucous membrane. Vasodilation allows for the leakage of fluid through the wall of the blood vessels into the bronchial mucous membrane, causing it to swell. This bronchiolar edema can prevent the orally-inhaled steroid from reaching the intracellular environment, thus delaying its onset of action. In order to enhance the activity of an orally inhaled steroid, a vasoconstrictor could be added to the bronchodilator/steroid combination. This additional agent would also have the added benefit of rapidly reducing the amount of mucosal edema, thereby enlarging the diameter of the airways. The patient would then get almost instantaneous relief from wheezing and shortness of breath as a result of the vasoconstrictor and bronchodilator activity. Unfortunately, vasoconstrictors such as oxymetazoline, if used for more than a few days, can cause a chemical irritation to the bronchiolar mucosa and a rebound phenomena. To counter this adverse effect, the vasoconstrictor could be administered with an inhaled steroid since the steroid would prevent both the chemical irritation and the rebound associated with prolonged use of vasoconstrictors.

The ideal regimen for the treatment of pulmonary disorders such as asthma would simultaneously address and provide rapid relief for all of the symptoms described above, including contraction of the muscles surrounding the airways, vasodilation of the mucosal blood vessels with resultant tissue edema that prevents the intracellular uptake of the orally inhaled steroid and narrows the airway and the inflammatory process involving the bronchiolar mucosa with the production of inspissated mucous. The present invention addresses these needs by providing for the concomitant administration of three different active agents: a bronchodilator, a vasoconstrictor and a corticosteroid.

### SUMMARY OF THE INVENTION

One aspect of the invention relates to a pharmaceutical formulation for pulmonary drug administration, comprising a therapeutically effective amount of at least one bronchodilator; a therapeutically effective amount of at least one vasoconstrictor; and a therapeutically effective amount of at least one corticosteroid.

Another aspect of the invention pertains to a method for treating a patient suffering from a pulmonary disorder, comprising administering to the patient, by inhalation, a pharmaceutical formulation for pulmonary drug administration, wherein the formulation comprises a therapeutically effective amount of at least one bronchodilator; a therapeutically effective amount of at least one vasoconstrictor; and a therapeutically effective amount of at least one corticosteroid.

Yet another aspect of the invention relates to a pulmonary drug delivery device, comprising: a pharmaceutical formulation comprised of a therapeutically effective amount of at least one bronchodilator, a therapeutically effective amount of at least one vasoconstrictor, and a therapeutically effective amount of at least one corticosteroid; and a means for housing and dispensing unit dosages of the formulation.

Still another aspect of the invention relates to a dry powder inhaler for orienting and positioning a capsule containing a pharmaceutical formulation to be administered by inhalation, comprising a dispensing chamber containing a capsule of a dry powder pharmaceutical formulation comprised of a therapeutically effective amount of at least one bronchodilator, a therapeutically effective amount of at least one vasoconstrictor, and a therapeutically effective amount of at least one corticosteroid, and a pharmaceutically acceptable carrier suitable for pulmonary drug administration; a tube for receiving the capsule to be oriented and dispensed; a ramp surface extending substantially across the tube from one wall to an opposite wall thereof; and an elongate dispensing passage having a diameter less than that of the tube and sized to receive the capsule only when the elongate axis of the capsule is generally parallel to the axis of the passage, the passage extending from an inlet end formed by an aperture in the ramp's surface to a dispensing outlet, the passage being adjacent to one wall of the tube such that the axis of the passage is parallel to, but radially offset from, an axis of the tube, whereby when the inhaler is positioned with the passage below the tube and the axis of the passage is substantially vertical, a capsule located in the tube is guided by the ramp surface towards the inlet end of the passage.

Yet another aspect of the invention relates to a dosage form containing a pharmaceutical composition for pulmonary drug administration, the pharmaceutical composition comprising a therapeutically effective amount of at least one bronchodilator, a therapeutically effective amount of at least one vasoconstrictor, and a therapeutically effective amount of at least one corticosteroid.

### BRIEF DESCRIPTION OF THE DRAWINGS

FIG. 1 is a cross-sectional side view of a preferred dry powder inhaler for administering the formulations of the invention.

FIG. 2 is a side view of the inhaler of FIG. 1, inverted so as to be positioned for delivering drug.

### DETAILED DESCRIPTION OF THE INVENTION

### I. Definitions

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a bronchodilator” includes a combination of two or more bronchodilators, reference to “a vasoconstrictor” includes combinations of two or more vasoconstrictors, reference to “a pharmaceutically acceptable carrier” includes combinations of two or more pharmaceutically acceptable carriers, and the like.

In describing and claiming the present invention, the following terminology will be used in accordance with the definitions set forth below.

The terms “active agent,” “drug” and “pharmacologically active agent” are used interchangeably herein to refer to a chemical material or compound which, when administered to an organism (human or animal) induces a desired pharmacologic effect. Included are derivatives which include pharmacologically acceptable and pharmacologically active salts, esters and amides, as well as prodrugs, conjugates and active metabolites. Analogs of those compounds or classes of compounds specifically mentioned that also induce the desired pharmacologic effect, are also included.

By “pharmaceutically acceptable carrier” is meant a material or materials that are suitable for pulmonary drug administration and not biologically or otherwise undesirable, i.e., that may be administered to an individual along with an active agent without causing any undesirable biological effects or interacting in a deleterious manner with any of the other components of the pharmaceutical formulation in which it is contained. Typically, the material (e.g., carrier or excipient) has met the required standards of toxicological and manufacturing testing or it is included on the Inactive Ingredient Guide prepared by the U.S. Food and Drug administration.

Similarly, a “pharmacologically acceptable” salt, ester, amide or other derivative of an active agent as provided herein is a salt, ester, amide or other derivative that is not biologically or otherwise undesirable.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using routine experimentation.

The term “dosage form” denotes any form of a pharmaceutical composition that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” asthma, as the term “treating” is used herein, encompasses both prevention of asthma in a predisposed individual and treatment of asthma in a clinically symptomatic individual.

The terms “condition,” “disease” and “disorder” are used interchangeably herein as referring to a physiological state that can be prevented or treated by administration of a pharmaceutical formulation as described herein.

The term “patient” as in treatment of “a patient” refers to a mammalian individual afflicted with or prone to a condition, disease or disorder as specified herein, and includes both humans and animals.

The term “pulmonary” as used herein refers to any part, tissue or organ that is directly or indirectly involved with gas exchange, i.e., O₂/CO₂ exchange, within a patient. “Pulmonary” contemplates both the upper and lower airway passages and includes, for example, the mouth, nose, pharynx, oropharynx, laryngopharynx, larynx, trachea, carina, bronchi, bronchioles and alveoli. Thus, the phrase “pulmonary drug administration” refers to administering the formulation described herein to any part, tissue or organ that is directly or indirectly involved with gas exchange within a patient.

“Optional” or “optionally” means that the subsequently described event or circumstance may or may not occur, and that the description includes instances where said event or circumstance occurs and instances where it does not. For example, reference to an “optional pharmaceutically acceptable carrier” in a formulation indicates that such a carrier may or may not be present, and the description includes formulations wherein a carrier is present and formulations wherein a carrier is not present.

### II. Pharmaceutical Formulations

The invention, as noted above, is in one embodiment a pharmaceutical formulation for pulmonary drug administration, comprising a therapeutically effective amount of at least one bronchodilator; a therapeutically effective amount of at least one vasoconstrictor; and a therapeutically effective amount of at least one corticosteroid. A pharmaceutically acceptable carrier suitable for pulmonary drug administration may also be included. Thus, the formulations described herein include at least three active agents, i.e., a bronchodilators, a vasoconstrictor and a corticosteroid.

The formulation of the present invention may also contain various excipients, provided such excipients do not have a deleterious effect on the intended patient or have a deleterious chemical or physical effect on any component in the formulation. Thus, for example, excipients such as preservatives, surface active agents, buffering agents, suspending agents, and the like can be combined with the formulation. The type and amount of any excipient will depend on the type of formulation and the device used for administration, as will be appreciated by one of ordinary skill in the art. Specific examples of each of these excipients are well known by those skilled in the art of pharmaceutical formulation.

The formulations of the present invention may take any form suitable for delivering the active agents to a patient. For example, the formulations may be in the form of a dry powder, aerosol or liquid.

A. Active Agents

Any of the active agents in the formulation may be administered in the form of a pharmacologically acceptable salt, ester, amide, prodrug or derivative or as a combination thereof. Salts, esters and derivatives of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol and the acid is added at a temperature of about 0 to 100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, or the like. Preparation of esters involves functionalization of hydroxyl and/or carboxyl groups that may be present within the molecular structure of the drug. The esters are typically acyl-substituted derivatives of free alcohol groups, i.e., moieties which are derived from carboxylic acids of the formula RCOOH where R is alkyl, and preferably is lower, i.e., C₁₋₆ alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

Stereoisomers of the active agents are also included as part of the formulations described herein. A stereoisomer is a compound having the same molecular weight, chemical composition, and constitution as another, but with the atoms arranged differently. That is, certain identical chemical moieties are at different orientations in space. This difference has the consequence of rotating the plane of polarized light. A pair of stereoisomers that are mirror images of each other are defined as enantiomers. Individual stereoisomers or enantiomers may have unique or beneficial properties that make that individual isomer particularly well suited for the present invention. Consequently, individual stereoisomers or enantiomers and mixtures thereof of the active agents are included as part of the invention. Thus, each active agent may be present in the formulation as a racemate, i.e., equal amounts of each enantiomer, an enantiomerically pure form, e.g., levalbuterol, or a mixture of nonequal amounts of each enantiomer, e.g., nonequal amounts of (S)-albuterol/(R)-albuterol.

The various hydrates of the active agents are also included in the formulations of the invention. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, exposing the active agent to dry heat.

1. Bronchodilators

The invention is not limited with respect to the bronchodilator. Furthermore, the formulation is not limited to one bronchodilator as combinations of bronchodilators may also be present. Bronchodilators from the pharmacological classes of alpha-adrenergic agonists, and beta-2 adrenergic agonists may be incorporated into the formulations. In a preferred embodiment, the bronchodilator is a beta-2 adrenergic agonist, which has agonist activity for β₂ adrenergic receptors.

Exemplary bronchodilators of the alpha adrenergic agonist class include, but are not limited to, ephedrine, epinephrine, isoproterenol, levarterenol, pseudoephedrine and phenylpropanolamine.

Exemplary bronchodilators of the beta-2 adrenergic agonist class include, but are not limited to, albuterol, bitolterol, fenoterol, formoterol, levalbuterol (i.e., homochiral (R)-albuterol), metaproterenol, pirbuterol, salmeterol, terbutaline, and derivatives thereof. Preferred beta-2 adrenergic agonists include formoterol, levalbuterol, metaproterenol, pirbuterol, salmeterol, and derivatives thereof.

The bronchodilator may be present in the formulation as a salt, ester, amide, prodrug, or other derivative, or may be functionalized in various ways as will be appreciated by those skilled in the art. Exemplary derivatives include albuterol sulfate, ipratropium bromide, levalbuterol hydrochloride, levalbuterol sulfate, metaproterenol sulfate, pirbuterol acetate, pirbuterol dihydrochloride, salmeterol xinafoate, and terbutaline sulfate. Depending on the particular derivative selected, certain formulations may be preferred over others. For example, levalbuterol sulfate is preferably formulated in a dry powder formulation, while levalbuterol hydrochloride is preferably formulated as an aerosol or liquid.

2. Vasoconstrictors

The invention is not limited with respect to the vasoconstrictor or vasopressor agent. Furthermore, the formulation is not limited to one vasoconstrictor as combinations of vasoconstrictors may also be present.

Typical vasoconstrictors include, but are not limited to, dobutamine, dopamine, ephedrine, epinephrine, ethylnorepinephrine, isoproterenol, methoxamine, naphazoline, norepinephrine, oxymetazoline, phenylephrine, phenylpropanolamine, propylhexedrine, pseudoephedrine, tetrahydrozoline, tramazoline, xylometazoline, xylometrazole, and derivatives thereof. Preferred vasoconstrictors include oxymetazoline and phenylephrine, and derivatives thereof.

The vasoconstrictor may be present in the formulation as a salt, ester, amide, prodrug, or other derivative, or may be functionalized in various ways as will be appreciated by those skilled in the art. Exemplary derivatives include oxymetazoline hydrochloride and phenylephrine hydrochloride.

3. Corticosteroids

The invention is not limited with respect to the corticosteroid. Furthermore, the formulation is not limited to one corticosteroid as combinations of such agents may also be present.

Typical corticosteroids include, but are not limited to, beclomethasone, budesonide, flunisolide, fluticasone, mometasone, triamcinolone, and derivatives thereof. Preferred corticosteroids include budesonide, mometasone, and derivatives thereof.

The corticosteroid may be present in the formulation as a salt, ester, amide, prodrug, or other derivative, or may be functionalized in various ways as will be appreciated by those skilled in the art. Exemplary derivatives include beclomethasone diproprionate, fluticasone propionate, triamcinolone acetonide, and esters (e.g., acetate form, thiophene ester form or furoate form) of mometasone.

B. Dry Powder Formulations

The dry powder formulations as described herein include, at a minimum, the bronchodilator, vasoconstrictor, and corticosteroid. Such dry powder formulations can be administered by pulmonary inhalation to a patient without the benefit of a carrier. Preferably, dry powders formulations that do not include a carrier are administered with the aid of, for example, a dry powder inhaler as described herein.

Preferably, however, the dry powder formulations described herein include one or more pharmaceutically acceptable carriers. Although any carrier suitable for pulmonary drug administration may be used, pharmaceutical sugars are particularly preferred for use as carriers in the present invention. Preferred pharmaceutical sugars include those selected from the group consisting of fructose, galactose, glucose, lactitol, lactose, maltitol, maltose, mannitol, melezitose, myoinositol, palatinite, raffinose, stachyose, sucrose, trehalose, xylitol, as well as hydrates thereof, and combinations of any of the foregoing. In preferred embodiments, lactose, e.g., lactose U.S.P., serves as the carrier when the formulation is a dry powder.

Once selected, each active agent or the active agents in combination are blended to form a substantially homogeneous powder mixture. Techniques involved with the preparation of such powders are well known in the art. Briefly stated, however, the preparation generally includes the steps of reducing the particle size of each active agent (again, alone or in combination), and blending. Of course, reducing the particle size of each active agent is not required when a commercially available product having a suitable particle size is used. Techniques for reducing the particle size include, for example, using mills such as an air-jet mill or a ball mill. The active agents should have a particle size diameter of between about 0.1 to 65 μm for pulmonary administration. It is preferred that the active agent particles are about 1 to 10 μm, more preferably about 2 to 5 μm in diameter.

Similarly, the particle size of the remaining components, e.g., carrier, excipient, etc., must be controlled as well. The same techniques described above for reducing the particle size of active agents may be used to reduce the particle size of the remaining components. Again, such techniques are not required when the component is available commercially in the desired particle size range. Preferably, the remaining components, particularly the carrier, have a particle size from about 30 to 100 μm in diameter, with sizes from about 30 to 70 μm being most preferred.

For any given particle size range, it is preferred that at least about 60%, more preferably at least about 70%, still more preferably at least about 85%, of the stated particles have a size within the stated or given range. It is most preferred, however that at least about 90% of the particles have the size in the stated or given range. For example, when a component is stated to have a particle size less than 10 μm, it is most preferred that at least 90% of the particles of that component have a particle size of less than 10 μm.

As noted above, some components of the formulation may be commercially available in the desired particle size range. For example, a preferred lactose product for use in some embodiments of the present invention is the PHARMATOSE™ 325 brand of lactose monohydrate available from DMV International, Veghel, The Netherlands. According to the manufacturer, 100% of the lactose particles have a particle size of less than 100 μm, and only 5 to 10% of the particles have a particle size of less than 32 μm. Furthermore, a minimum of 70% of the lactose particles are stated to have a particle size of less than 63 μm. Advantageously, particle size manipulation steps are avoided when components are commercially available in the desired particle size range.

Preferably, the particle size reduction of the active agents and the particle size reduction of the remaining components are carried out separately. In this way, it is possible to provide a formulation in which the particle size of the active agents is smaller than the particle size of, for example, the carrier. The advantage of such a formulation is that the active agents penetrate deeply into the pulmonary tract while the carrier (having a relatively larger particle size) is retained in the upper airways.

Conventional blending techniques known to those skilled in the art may be used for combining active agents or for combining the active agents with the carrier and/or remaining components. Such blending techniques include passing the combined powders through a sifter or blending, for example, the active agents and carrier in a powder blender such as a “double cone” blender or a “V-blender.” No matter which technique is employed, however, it is necessary that the resulting powder is a substantially homogeneous mixture. Typically, the active agents will make up from about 0.01 to 99 wt % of the total formulation, preferably from about 0.05 to 50 wt % of the total formulation.

After blending, the powder formulation may, if desired, be portioned and/or otherwise processed into unit dose quantities, e.g., portioned into unit dose quantities and individually placed within a unit dosage form or drug delivery system. Alternatively, the powder formulation may be loaded into a dosage form or drug delivery device and not “metered out” into unit doses until used. Accordingly, the invention encompasses dosage forms containing a pharmaceutical composition for pulmonary drug administration comprising therapeutically effective amounts of at least one bronchodilator, at least one vasoconstrictor, and at least one corticosteroid.

Although any dosage form that contains a unit dose of the formulation is acceptable, vials and capsules are preferred, with capsules being most preferred. The capsule material may be either hard or soft, and, as will be appreciated by those skilled in the art, typically comprises a water-soluble compound such as gelatin, starch or a cellulosic material. Preferably, the capsules are composed of a cellulosic material, e.g., hydroxypropyl methylcellulose. The capsules may be sealed, such as with gelatin bands or the like. See, for example, Remington: The Science and Practice of Pharmacy, 20^{th} edition (Lippincott Williams & Wilkins, 2000), which describes materials and methods for preparing encapsulated pharmaceuticals. Thus, each capsule or dosage form will typically contain a therapeutically effective dose of each active agent. Alternatively, the dosage forms may contain less than a therapeutically effective dose in which case administration of two or more dosage forms would be used to provide the therapeutically effective dose.

C. Aerosol Formulations

The formulations of the present invention may also take the form of an aerosol composition for inhalation. Aerosol formulations are known to those skilled in the art and are described in Remington: The Science and Practice of Pharmacy, supra. Briefly, the aerosol formulation of the invention is either a solution aerosol in which the active agents are soluble in the carrier (e.g., propellant) and optional solvent or a dispersion aerosol in which the active agents are suspended or dispersed throughout the carrier and optional solvent. In a preferred embodiment, the aerosol formulations of the invention are in the form of a dispersion aerosol.

The carrier in the aerosol formulations of the invention is generally a propellant, usually a compressed gas, e.g., air, nitrogen, nitrous oxide, and CO₂, a mixture of compressed gases, a liquefied gas or a mixture of liquefied gases. A mixture of propellants, when present in the formulations, may be comprised of two, three, four or propellants. Preferred mixtures of propellants, however, comprise only two propellants. Any propellant used in the art of preparing aerosol formulations may be used.

Typically, the propellant is a chlorofluorocarbon, a hydrochlorofluorocarbon, a hydrogen-containing fluorocarbon, a perfluorocarbon, a hydrocarbon or a mixture thereof. Preferably, the propellant is a hydrochlorofluorocarbon, a hydrogen-containing fluorocarbon, a perfluorocarbon or a mixture thereof.

Preferred chlorofluorocarbons include dichlorotetrafluoroethanes (e.g., CClF₂CClF₂ and CCl₂FCF₃), trichloromonofluoromethane, dichlorodifluoromethane, chloropentafluoroethane, and mixtures thereof Preferred hydrochlorofluorocarbons include monochlorodifluoromethane, monochlorodifluoroethane (e.g., 1-chloro-1,1-difluoroethane), and mixtures thereof. Preferred hydrogen-containing fluorocarbons include C₁₋₄ hydrogen-containing fluorocarbons such as CHF₂CHF₂, 1,1,1,2-tetrafluoroethane (HFA-134a), difluoroethane (e.g., 1,1-difluoroethane), 1,1,1,2,3,3,3-heptafluoropropane (HFA-227), and mixtures thereof. Preferred perfluorocarbons include CF₃CF₃, CF₃CF₂CF₃, octafluorocyclobutane, and mixtures thereof. Preferred hydrocarbons include propane, isobutane, n-butane, dimethyl ether, and mixtures thereof. Most preferably, the propellant is selected from the group consisting of difluoroethane, CHF₂CHF₂, 1,1,1,2-tetrafluoroethane, 1,1,1,2,3,3,3-heptafluoropropane, CF₃CF₃, CF₃CF₂CF₃, octafluorocyclobutane, and mixtures of any of the foregoing.

As will be appreciated by one skilled in the art, the aerosol formulations of the invention may include one or more excipients. For example, the aerosol formulations may contain: a solvent (e.g., water, ethanol and mixtures thereof) for increasing the solubility of the active agent; an antioxidant (e.g., ascorbic acid) for inhibiting oxidative degradation of the active agents; a dispersing agent (e.g., sorbitan trioleate, oleyl alcohol, oleic acid, lecithin, e.g., soya lecithin, corn oil, or combinations thereof) for preventing agglomeration of particles; and/or a lubricant (e.g., isopropyl myristate) for providing slippage between particles and lubricating the components, e.g., the valve and spring, of the inhaler.

As described with respect to dry powder formulations, the particle size released from aerosol formulations must be appropriate for pulmonary administration. Solution aerosols inherently produce small particles upon actuation of the inhaler given that the active agents are expelled along with the carrier, i.e., propellant, solution as it evaporates. Consequently, solution aerosols produce sufficiently small particles, e.g., within a range of about 0.1 to about 65 μm, of active agents upon administration. In contrast, dispersion aerosols contain undissolved active agents in which particle size remains constant, i.e., the size of the particles in the dispersion aerosol remains unchanged as the active agent is delivered to the patient. Thus, the active agents must have an appropriate particle size before being formulated into a dispersion aerosol. Consequently, methods of reducing the particle size of the active agents for the dry powder formulations described above are equally applicable for preparing active agents with an appropriate particle size in a dispersion aerosol. Furthermore, the same ranges of particle sizes preferred for the dry powder formulations are equally applicable for dispersion aerosols.

The aerosol formulation may be prepared by employing a cold filling process. Initially, the components of the aerosol formulation and an aerosol container are cooled, e.g., to about −40° C., such that the carrier, i.e., propellant, is a liquid. All components except for the carrier are placed into the aerosol container. Thereafter, the carrier is added, the components mixed, and a valve assembly inserted into place. The valve assembly is then crimped such that the container is airtight. Thereafter, the container and formulation contained therein are allowed to return to ambient temperature.

As an alternative to the cold filling process, the aerosol formulation may be prepared by transfer of a carrier from a bulk container. In such a process, the components except for the carrier are initially placed into an empty aerosol container. A valve assembly is then inserted and crimped into place. The carrier, under pressure and in liquid form, is metered through the valve assembly from a bulk container or tank of carrier. The container housing the formulation is checked to ensure that the pressurized contents do not leak.

For both of these methods of preparing the aerosol formulations, the active agents generally represent from about 0.1 to 40 wt. % of the total formulation. In one preferred embodiment, the active agents represent about 2 to 20 wt. % of the total formulation, with 5 to 15 wt. % being more preferred.

D. Liquid Formulations

The formulations of the present invention may also take the form of a liquid composition for inhalation. Liquid formulations are well known in the art. See, for example, Remington: The Science and Practice of Pharmacy, supra. It is preferred that the liquid is an aqueous suspension, although aqueous solutions may be used as well. The liquid formulations include one or more carriers in addition to the active agents. Generally, the carrier is a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Combining the components followed by conventional mixing results in a liquid formulation suitable for inhalation. Typically, the active agents will make up from about 0.01 to 40% of the total formulation.

### III. Utility and Administration

The invention provides a method for treating a patient suffering from a pulmonary disorder, comprising administering to the patient, by inhalation, a pharmaceutical formulation for pulmonary drug administration, wherein the formulation comprises a therapeutically effective amount of at least one bronchodilator; a therapeutically effective amount of at least one vasoconstrictor; and a therapeutically effective amount of at least one corticosteroid.

Exemplary pulmonary disorders for which this method finds utility, include asthma (including exercised-induced asthma) and chronic obstructive pulmonary disease. The formulations are effective in the treatment of patients suffering from both acute and chronic episodes of these maladies.

A particularly preferred combination is levalbuterol, oxymetazoline and budesonide. All three active agents are readily available in aqueous form and can be easily mixed together and the mixture delivered by means of a nebulizer. This combination could be administered to patients suffering from sever asthma who are dependent on oral steroids, for example, they require oral dosages of prednisone to control wheezing and shortness of breath. By using the combination therapy of the invention, patients are expected to experience rapid and prolonged relief of these symptoms. In addition, it is expected that such patients can also be weaned off their dependence on oral prednisone, thus providing an additional advantage over the current therapies.

The formulations as described herein have many advantages over conventional inhalation formulations. Because the pharmaceutical formulation combines three active agents, patients receive the benefits of three active agents with only one formulation. As a further consequence of combining three active agents, the formulation of the present invention increases patient compliance as the likelihood of missed doses of a second and a third active agent is eliminated.

Furthermore, particular ingredients may have additional advantages when present in the formulation. For example, pirbuterol and levalbuterol do not cause side effects such as excitability and heart tremors that may be caused by other bronchodilators. Finally, the patient may only need to administer the formulation on an “as-needed” basis. For example, for patients suffering from asthma, the bronchodilator acts to relieve symptoms immediately while the vasoconstrictor component acts to reduce the amount of mucosal edema and bronchorrhea, and the steroidal antiinflammatory component acts to treat the underlying inflammation causing the asthmatic symptom(s). In addition, by reducing the amount of the mucusal edema and bronchorrhea, the vasoconstrictor also serves to enhance the uptake of the inhaled steroid. In this way, the formulation of the invention addresses both the symptoms and causes of an asthmatic attack, thereby obviating the need to administer repeated doses of a drug that only treats the symptoms of an asthmatic attack. Consequently, daily administration may not be necessary.

Additional advantages result when the formulations are administered by dry powder inhalers, which assure that patients, particularly those patients that have traditionally had trouble using inhalers such as children or the elderly, obtain the complete dose. Administration of the complete dose can be ensured with the dry powder inhalers described herein since little effort in inhalation is required in order to deliver all of the dose to the lungs. This is in contrast to, for example, metered-dose inhalers with which patients must coordinate the actuation of the inhaler with a deep and prolonged inhalation to ensure that the entire dose is received. As a result of the foregoing advantages, the dry powder inhalers described herein may be efficient in delivering the present formulations in reduced dosages, i.e., about 5 to 15% less than the dose used in conventional devices.

The actual amount of each active agent in the formulation will, of course, depend upon the age, weight, and general condition of the subject, the severity of the condition being treated, and the judgment of the prescribing physician. Therapeutically effective amounts of all three actives are known to those skilled in the art and/or are described in the pertinent reference texts and literature. An effective amount of the formulation may be administered with a single administration, e.g., serially administration of the contents of a single capsule containing a therapeutically effective amount of the formulation by a dry powder inhaler or a single actuation of an aerosol inhaler designed to deliver a therapeutically effective amount of the formulation. Alternatively, a patient can obtain an effective amount of the formulation by, for example, administering multiple doses, e.g., serially administering the contents of multiple capsules containing the formulation by a dry powder inhaler.

For those active agents that have known pharmacological profiles and dosing regimens (e.g., as referenced in the Physician's Desk Reference), one skilled in the art can readily determine appropriate dosages for use in the methods and compositions of the invention.

For those active agents for which dosing regimens are not known, determination of a therapeutically effective amount for any particular bronchodilator, vasoconstrictor, or corticosteroid is well within the capability of those skilled in the art. That is, for any of the present active agents, a therapeutically effective dose can be estimated initially from cell culture assays. For example, a dose can be formulated to achieve a circulating concentration range that includes an IC₅₀ value as determined in cell culture (i.e., the concentration of the test compound required to reduce enzyme activity by 50%). Such information can be used to more accurately determine useful doses in humans. In addition, toxicity and therapeutic efficacy of any given active agent can be determined by standard pharmaceutical procedures in cell cultures or experimental animals, e.g., procedures used for determining the maximum tolerated dose (MTD), the ED₅₀, which is the effective dose to achieve 50% of maximal response, and the therapeutic index (TI), which is the ratio of the MTD to the ED₅₀. Obviously, compounds with high TIs are the most preferred drugs, and preferred dosage regimens are those that maintain plasma levels of the active agent at or above a minimum concentration to maintain the desired therapeutic effect.

Furthermore, the actual amount of each active agent will also depend on particular “synergies” between any two active agents or all three active agents. That is, certain combinations and/or ratios of the bronchodilator/vasoconstrictor/corticosteroid combinations described herein provide enhanced treatment of a particular condition.

For the bronchodilator, for example, the formulation will be prepared such that each dose (or administration) of the formulation will deliver the bronchodilator in a therapeutically effective amount, typically in the range of about 1 to 1500 μg. When the bronchodilator is pirbuterol acetate or pirbuterol dihydrochloride, for example, a suitable dosage is in the range of about 2.5 to 350 μg, preferably from about 50 to 100 μg. When the bronchodilator is levalbuterol sulfate, a suitable dosage is in the range of about 5 to 150 μg, preferably from about 50 to 100 μg. When the bronchodilator is levalbuterol hydrochloride, a suitable dose is in the range of about 50 to 1300 μg, preferably from about 600 to 1000 μg.

For the vasoconstrictor, for example, the formulation will be prepared such that each dose (or administration) of the formulation will deliver the vasoconstrictor in a therapeutically effective amount, typically in the range of about 10 to

μg. When the vasoconstrictor is oxymetazoline, for example, a suitable dosage is in the range of about 50 to 1000 μg, preferably from about 100 to 500 μg. When the vasoconstrictor is phenylephrine, for example, a suitable dosage is in the range of about 500 to 10,000 μg, preferably from about 1000 to 5000 μg.

For the corticosteroid, for example, the formulation will be prepared such that each dose (or administration) of the formulation will deliver the corticosteroid in a therapeutically effective amount, typically in the range of about 1 to 2000 μg. When the corticosteroid is budesonide, for example, a suitable dosage is in the range of about 100 to 2000 μg, preferably from about 200 to 800 μg. When the corticosteroid is anhydrous mometasone furoate or mometasone furoate monohydrate, a suitable dosage is within the range of about 1 to 1500 μg, preferably from about 50 to 200 μg. When the corticosteroid is fluticasone, for example, a suitable dosage is in the range of about 44 to 1000 μg, preferably from about 88 to 880 μg. When the corticosteroid is triamcinolone, for example, a suitable dose is in the range of about 100 to 1600 μg, preferably from about 200 to 800 μg.

The formulations may be administered in a variety of dosing regimens including: as-needed administration; one, two, three or four administrations once daily; one, two, three or four administrations twice daily; one, two, three or four administrations three times daily; and one, two, three or four administrations four times daily. Generally, however, the total daily dose of the bronchodilator should not exceed about 5000 μg, the total daily dose of the vasoconstrictor should not exceed about 10,000 μg, and the total daily dose of the corticosteroid should not exceed about 8000 μg.

The formulations of the invention may be administered by oral or nasal inhalation. For oral administration, the patient inhales the formulation through the mouth. The inhaled formulation progressively comes into contact with the air passages of the mouth and throat area, the upper respiratory tract, e.g., trachea, and finally the lower respiratory tract, e.g., bronchioles. The bronchodilator, e.g., β₂ agonist, acts to relax bronchial smooth muscle, which also facilitates gas exchange by opening up closed or constricted passages. The vasoconstrictor, acts to reduce the amount of mucosal edema. The corticosteroid acts to decrease inflamed and congested air passages, thereby facilitating gas exchange by increasing the diameter of the air passages.

Nasal inhalation is similar to oral inhalation except that the patient inhales the formulation through the nares, either one at a time or simultaneously. For example, the formulation may be administered by a pump spray in which the patient administers a spray or powder in the left nare followed by administration in the right nare, or by using a powder inhaled in both nares simultaneously. Nasal administration provides an added benefit of relieving nasal congestion (if present) in that the vasoconstrictor and corticosteroid are placed in contact with nasal tissue since the vasoconstrictor reduces the amount of tissue edema and the amount of rhinorrhea, thus enhancing the absorption of the corticosteroid.

### IV. Inhalers

The invention also provides a dry powder inhaler containing a formulation as described herein. Dry powder inhalers are well known to those skilled in the art. Preferably, the dry powder inhaler includes at least one capsule (preferably a hydroxypropyl methylcellulose capsule) containing a unit dose of the formulation. The patient self-administers the dose by inhaling (by oral or nasal inhalation) the dry powder formulation from the inhaler. In this manner, delivery of the dry powder formulation to the pulmonary system is effected. Accordingly, one embodiment of the invention encompasses a pulmonary drug delivery device that comprises a pharmaceutical formulation containing therapeutically effective amounts of at least one bronchodilator, at least one vasoconstrictor, and at least one corticosteroid. The device also includes a means for housing and dispensing unit dosages of the formulation.

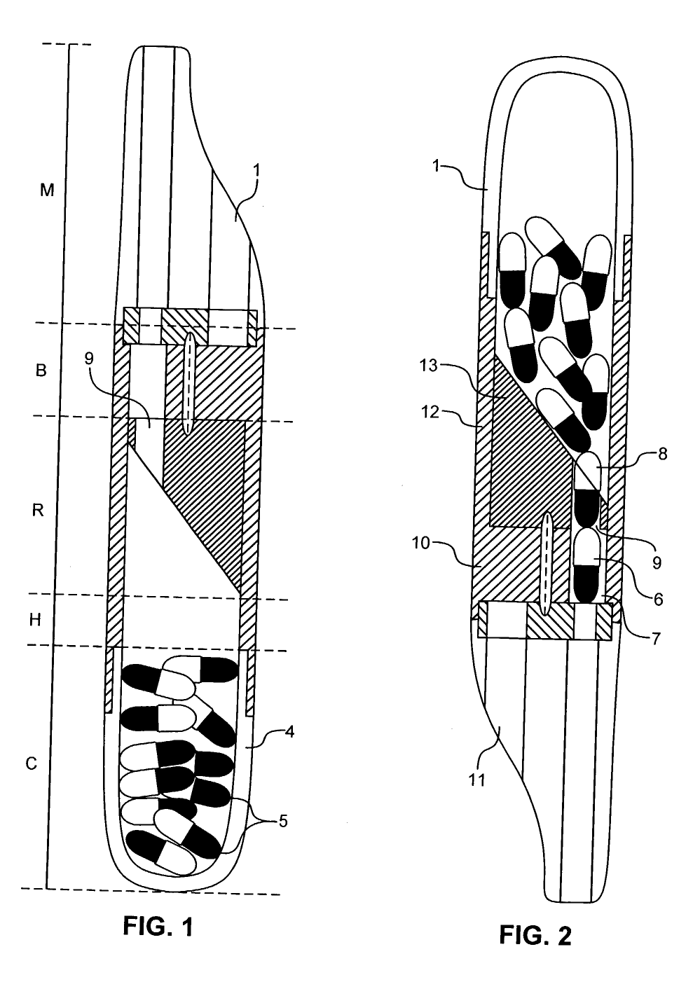

One example of a particularly preferred dry powder inhaler is described in U.S. Pat. No. 5,673,686 to Villax et al. and U.S. Pat. No. 5,881,721 to Bunce et al. Specifically, as shown in FIG. 1, a dry powder inhaler 1 comprises a mouthpiece M, a barrel area B, a ramp area R, free headspace H and a capsule container area C. The capsule container 4 is filled to the brim with capsules 5. FIG. 2 shows the same inhaler 1 which has been inverted. The capsules now fill the free headspace and the ramp area and become vertically oriented as they near the passage 9. One capsule 8 is already inserted into the passage 9 and its movement is blocked by the capsule 6 which has preceded it and been dispensed into the capsule chamber 7. The capsule chamber 7 is contained inside a rotating barrel 10.

The operation of the inhaler requires that once a capsule has been loaded into the capsule chamber 7, the rotating barrel 10 is turned. This movement transports the capsule 6 past two small blades (not shown), which slits both ends and carries the capsule to the inhalation position. Once inhalation has taken place, a further turn of the barrel 10 delivers the capsule to the ejection position 11. Continuing to turn the rotating barrel 10 brings the capsule chamber 7 in alignment again with the passage 9 where the next capsule 8 is in place for dispensing.

The rotating barrel 10 is connected to the cylindrical tube 12 and is unconnected to the ramp 13. In operation, the turning motion of the rotating barrel 10 and cylindrical tube 12 is in opposite direction to that of the ramp 13. These opposite turning motions further assist the righting of the capsules between the ramp 13 and the cylindrical tube 12 and dispensing of the capsule into the passage 9.

Thus, the dry powder inhaler comprises a tube, a ramp, and a dispensing passage. The tube receives a capsule or similar dosage unit that must be properly oriented. The ramp has a surface that extends substantially across the tube from one wall to an opposite wall. An elongate dispensing passage has a diameter less than that of the tube and is sized to receive the capsule to be dispensed, but only when the axis of the capsule is generally parallel to the axis of the passage. The elongate dispensing passage extends from an inlet end formed by an aperture in the ramp's surface to a dispensing outlet, the passage being adjacent to one wall of the tube such that the axis of the passage is parallel to, but radially offset from, an axis of the tube. The arrangement is such that when the apparatus is positioned with the passage below the tube and the axis of the passage is substantially vertical, a capsule located in the tube will be guided by the ramp surface towards the inlet end of the passage.

Once the capsule has been properly aligned and pierced, the patient inserts the end of the mouthpiece of the inhaler into his or her mouth and inhales. Air enters through the device by any path but generally though specialized air inlets (not shown) on the device. As air enters the inhaler, at least a portion is drawn through an upstream slit. As it travels through the upstream slit into the pierced capsule, the air fluidizes or entrains the powder in the pierced capsule creating what has been referred to as a “dancing cloud.” As suction continues from the patient, the powder-containing air exits through a downstream slit in the pierced capsule and enters the bore of the mouthpiece for passage into the patient's pulmonary system.

Accordingly, one preferred embodiment of the invention is a dry powder inhaler for orienting and positioning a capsule containing a pharmaceutical formulation to be administered by inhalation. The inhaler has a dispensing chamber containing a capsule of a dry powder pharmaceutical formulation comprised of a therapeutically effective amount of at least one bronchodilator, a therapeutically effective amount of at least one vasoconstrictor, and a therapeutically effective amount of at least one corticosteroid, and a pharmaceutically acceptable carrier suitable for pulmonary drug administration; a tube for receiving the capsule to be oriented and dispensed; a ramp surface extending substantially across the tube from one wall to an opposite wall thereof; and an elongate dispensing passage having a diameter less than that of the tube and sized to receive the capsule only when the elongate axis of the capsule is generally parallel to the axis of the passage, the passage extending from an inlet end formed by an aperture in the ramp's surface to a dispensing outlet, the passage being adjacent to one wall of the tube such that the axis of the passage is parallel to, but radially offset from, an axis of the tube, whereby when the inhaler is positioned with the passage below the tube and the axis of the passage is substantially vertical, a capsule located in the tube is guided by the ramp surface towards the inlet end of the passage.

Other dry powder inhalation devices suitable for administering the present invention include, for example, Turbuhaler® (Astra Pharmaceutical Products, Inc., Westborough, Mass.), Rotahaler® and Diskhaler® devices (both available from Allen & Hanburys, Ltd., London, England). Aerosol formulations of the present invention may be administered by pressurized metered-dose inhalers. Liquid formulations of the invention may be administered by a pump spray bottle or nebulizer.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### Experimental

The practice of the present invention will employ, unless otherwise indicated, conventional techniques of pharmaceutical formulation, medicinal chemistry and the like, which are within the skill of the art. Such techniques are explained fully in the literature. Preparation of various types of pharmaceutical formulations are described, for example, in Remington: The Science and Practice of Pharmacy, 20^{th} edition (Lippincott Williams & Wilkins, 2000) and Ansel et al., Pharmaceutical Dosage Forms and Drug Delivery Systems, 6^{th} Ed. (Media, P A: Williams & Wilkins, 1995).

The following examples are put forth so as to provide those of ordinary skill in the art with a complete disclosure and description of how to make and use the compositions of the invention. Efforts have been made to ensure accuracy with respect to numbers (e.g., amounts, temperature, etc.) but some experimental error and deviations should, of course, be allowed for. Unless indicated otherwise, parts are parts by weight, temperature is degrees centigrade and pressure is at or near atmospheric. All components were obtained commercially unless otherwise indicated.

### EXAMPLE 1

Levalbuterol sulfate (10.0 mg), 10.0 mg of budesonide, 10.0 mg of oxymetazoline hydrochloride, and 2000 mg of lactose are blended using conventional blending techniques to form a dry pharmaceutical formulation. Particle size reduction is not required as each of the components is obtained having a suitable particle size. The dry pharmaceutical formulation is then divided, in equal portions, into 100 capsules (capsule size 4). The amount of each component of the formulation per capsule is presented below.

| Component | Amount per capsule |
|---|---|
| levalbuterol sulfate | 100 μg |
| budesonide | 100 μg |
| oxymetazoline hydrochloride | 100 μg |
| lactose | 20.00 mg |

### EXAMPLE 2

Levalbuterol sulfate (20.0 mg), 20.0 mg of budesonide, 20.0 mg of oxymetazoline hydrochloride, and 2500 mg of lactose are blended using conventional blending techniques to form a dry pharmaceutical formulation. The dry pharmaceutical formulation is then divided, in equal portions, into 100 capsules (capsule size 4). The amount of each component of the formulation per capsule is presented below.

| Component | Amount per capsule |
|---|---|
| levalbuterol sulfate | 200 μg |
| budesonide | 200 μg |
| oxymetazoline hydrochloride | 200 μg |
| lactose | 25.00 mg |

### EXAMPLE 3

Levalbuterol hydrochloride (75.0 mg), 10.0 mg of mometasone furoate (anhydrous), 10.0 mg of oxymetazoline hydrochloride, and 2500 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented below.

| Component | Amount per capsule |
|---|---|
| levalbuterol hydrochloride | 750 μg |
| mometasone furoate (anhydrous) | 100 μg |
| oxymetazoline hydrochloride | 100 μg |
| lactose | 25.00 mg |

### EXAMPLE 4

Pirbuterol acetate (20.0 mg), 20.0 mg of mometasone furoate (anhydrous), 20.0 mg of phenylephrine hydrochloride, and 2500 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented below.

| Component | Amount per capsule |
|---|---|
| pirbuterol acetate | 200 μg |
| mometasone furoate (anhydrous) | 200 μg |
| phenylephrine hydrochloride | 200 μg |
| lactose | 25.00 mg |

### EXAMPLE 5

The capsules made in any one of Examples 1 through 4 are placed in the dry powder inhaler as described in U.S. Pat. No. 5,673,686 to Villax et al. and U.S. Pat. No. 5,881,721 to Bunce et al. and as illustrated in FIG. 1 and FIG. 2. Once the capsule has been properly aligned and pierced in the inhaler, a patient having an asthmatic attack inserts the mouthpiece of the inhaler into their mouth and inhales normally through the mouth. The inhalation causes the formulation to exit the pierced capsule and travel into the patient's pulmonary system. Relief of the asthmatic attack is expected to occur immediately.

### EXAMPLE 6

A liquid suspension for inhalation is prepared. The percent amount of each component of the formulation is presented below.

| | Amount % |
|---|---|
| Component | (wt./wt.) |
| pirbuterol acetate | 0.20% |
| mometasone furoate monohydrate | 0.05%* |
| oxymetazoline hydrochloride | 0.05% |
| glycerin | 2.1% |
| microcrystalline cellulose and carboxymethylcellulose | 2.0% |
| sodium mixture, e.g., Avicel ® RC-591 (available | |
| from FMC, Corp., Philadelphia, PA) | |
| sodium citrate | 0.28% |
| phenylethyl alcohol | 0.25% |
| citric acid | 0.20% |
| benzalkonium chloride | 0.20% |
| oleate ester of sorbitol and its anhydride | 0.01% |
| copolymerized with ethylene oxide, e.g., | |
| polysorbate 80 (available from ICI Americas, | |
| Bridgewater, NJ) | |
| Water | q.s. |

*Equivalent to mometasone furoate calculated on the anhydrous basis.

Microcrystalline cellulose and the carboxymethylcellulose sodium mixture are dispersed in water followed by the addition of glycerin to form a dispersion. A solution of citric acid and sodium citrate in water is prepared and then added to dispersion. Separately, oleate ester of sorbitol and its anhydride copolymerized with ethylene oxide is dissolved in water and stirred. Pirbuterol acetate, mometasone furoate monohydrate, and oxymetazoline hydrochloride are added to the sorbitol solution and mixed to form a slurry. The slurry is then added to the dispersion with simultaneous stirring to form a suspension. Both the benzalkonium chloride and phenylethyl alcohol are dissolved in water and then added to the suspension with simultaneous stirring. Water is added to bring the suspension to 100%.

The liquid suspension is administered to a patient via a conventional pump spray bottle adapted for nasal inhalation. Following administration, the patient is expected to notice a decrease of allergy-induced bronchospasms.

### EXAMPLE 7

Example 6 is followed except that levalbuterol sulfate (0.09 wt./wt. %) is substituted for pirbuterol acetate.

### EXAMPLE 8

A metered-dose inhaler is prepared. The percent amount of each component in the formulation is presented below.

| Component | Amount per capsule |
|---|---|
| pirbuterol acetate | 5-15% |
| mometasone furoate (anhydrous) | 5-15% |
| xylometrazole | 5-15% |
| fluorinated hydrocarbon | 70-90% |
| (propellant/carrier) | |

The active components are placed into an empty aerosol container. Thereafter, a valve assembly is inserted into the aerosol container and crimped into place so as to provide an airtight seal. The propellant/carrier is then metered through the valve assembly from a tank of bulk propellant/carrier stored under pressure. The aerosol container is then placed in an adaptor suited for actuating aerosol containers and delivering metered amounts of the active agents to a patient. An asthmatic patient is expected to be relieved of their asthmatic symptoms upon pulmonary administration of the formulation via the metered-dose inhaler.

I claim:

1. A pharmaceutical formulation for pulmonary drug administration, comprising:
a therapeutically effective amount of at least one bronchodilator;
a therapeutically effective amount of at least one vasoconstrictor; and
a therapeutically effective amount of at least one corticosteroid.

2. The formulation of claim 1, wherein the bronchodilator is selected from the group consisting of alpha-adrenergic agonists, beta-2 adrenergic agonists, and combinations thereof.

3. The formulation of claim 2, wherein the bronchodilator is an alpha adrenergic agonist selected from the group consisting of ephedrine, epinephrine, isoproterenol, levarterenol, pseudoephedrine, phenylpropanolamine, and derivatives thereof.

4. The formulation of claim 2, wherein the bronchodilator is a beta-2 adrenergic agonist.

5. The formulation of claim 4, wherein the beta-2 adrenergic agonist is selected from the group consisting of albuterol, bitolterol, fenoterol, formoterol, levalbuterol (i.e., homochiral (R)-albuterol), metaproterenol, pirbuterol, salmeterol, terbutaline, and derivatives thereof.

6. The formulation of claim 5, wherein the beta-2 adrenergic agonist is selected from the group consisting of formoterol, levalbuterol, metaproterenol, pirbuterol, salmeterol, and derivatives thereof.

7. The formulation of claim 1, wherein the vasoconstrictor is selected from the group consisting of dobutamine, dopamine, ephedrine, epinephrine, ethylnorepinephrine, isoproterenol, methoxamine, naphazoline, norepinephrine, oxymetazoline, phenylephrine, phenylpropanolamine, propylhexedrine, pseudoephedrine, tetrahydrozoline, tramazoline, xylometazoline, xylometrazole, and derivatives thereof.

8. The formulation of claim 7, wherein the vasoconstrictor is selected from the group consisting of oxymetazoline and phenylephrine, and derivatives thereof.

9. The formulation of claim 1, wherein the corticosteroid is selected from the group consisting of beclomethasone, budesonide, flunisolide, fluticasone, mometasone, triamcinolone, and derivatives thereof.

10. The formulation of claim 9, wherein the corticosteroid is budesonide, mometasone, or a derivative thereof.

11. The formulation of claim 1, wherein the bronchodilator is levalbuterol, the vasoconstrictor is oxymetazoline and the corticosteroid is budesonide, or derivatives thereof.

12. The formulation of claim 1, which further comprises a pharmaceutically acceptable carrier suitable for pulmonary drug administration.

13. The formulation of claim 1, in the form of a dry powder.

14. The formulation of claim 13, which further comprises a pharmaceutically acceptable carrier selected from the group consisting of fructose, galactose, glucose, lactitol, lactose, maltitol, maltose, mannitol, melezitose, myoinositol, palatinite, raffinose, stachyose, sucrose, trehalose, xylitol, hydrates and combinations thereof.

15. The formulation of claim 14, wherein the carrier is lactose.

16. The formulation of claim 1, in the form of an aerosol composition.

17. The formulation of claim 16, which further comprises a pharmaceutically acceptable carrier selected from the group consisting of a chlorofluorocarbon, a hydrochlorofluorocarbon, a hydrogen-containing fluorocarbon, a perfluorocarbon, a hydrocarbon, and mixtures thereof.

18. The formulation of claim 17, wherein the carrier is selected from the group consisting of dichlorotetrafluoroethanes, trichloromonofluoromethane, dichlorodifluoromethane, chloropentafluoroethane, monochlorodifluoromethane, monochlorodifluoroethane, difluoroethane, CHF₂CHF₂, 1,1,1,2-tetrafluoroethane, 1,1,1,2,3,3,3-heptafluoropropane, CF₃CF₃, CF₃CF₂CF₃, octafluorocyclobutane, propane, isobutane, n-butane, dimethyl ether, and mixtures thereof.

19. The formulation of claim 18, wherein the carrier is selected from the group consisting of difluoroethane, CHF₂CHF₂, 1,1,1,2-tetrafluoroethane, 1,1,1,2,3,3,3-heptafluoropropane, CF₃CF₃, CF₃CF₂CF₃, octafluorocyclobutane, and mixtures thereof.

20. The formulation of claim 1, in the form of a liquid.

21. The formulation of claim 20, wherein the liquid is an aqueous suspension.

22. The formulation of claim 20, which further comprises a sodium chloride solution.

23. The formulation of claim 1, in a unit dosage form.

24. The formulation of claim 23, wherein the unit dosage form is a hydroxypropyl methylcellulose capsule.

25. The formulation of claim 23, wherein the unit dosage form is a unit dose vial.

26. The formulation of claim 23, wherein the therapeutically effective amount of the bronchodilator is within the range of about 1 to 1500 μg.

27. The formulation of claim 23, wherein the therapeutically effective amount of the vasoconstrictor is within the range of about 10 to 10,000 μg.

28. The formulation of claim 23, wherein the therapeutically effective amount of the corticosteroid is within the range of about 1 to 2000 μg.

29. A pharmaceutical formulation for pulmonary drug administration, comprising:
a therapeutically effective amount of a bronchodilator selected from the group consisting of albuterol, bitolterol, fenoterol, formoterol, levalbuterol (i.e., homochiral (R)-albuterol), metaproterenol, pirbuterol, salmeterol, terbutaline, and derivatives thereof;
a therapeutically effective amount of a vasoconstrictor selected from the group consisting of dobutamine, dopamine, ephedrine, epinephrine, ethylnorepinephrine, isoproterenol, methoxamine, naphazoline, norepinephrine, oxymetazoline, phenylephrine, phenylpropanolamine, propylhexedrine, pseudoephedrine, tetrahydrozoline, tramazoline, xylometazoline, xylometrazole, and derivatives thereof;
a therapeutically effective amount of a corticosteroid selected from the group consisting of beclomethasone, budesonide, fluticasone, mometasone, triamcinolone, and derivatives thereof; and
lactose.

30. A method for treating a patient suffering from a pulmonary disorder, comprising administering to the patient, by inhalation, a pharmaceutical formulation for pulmonary drug administration, wherein the formulation comprises:
a therapeutically effective amount of at least one bronchodilator;
a therapeutically effective amount of at least one vasoconstrictor; and
a therapeutically effective amount of at least one corticosteroid.

31. The method of claim 30, which further comprises a pharmaceutically acceptable carrier suitable for pulmonary drug administration.

32. The method of claim 30, wherein inhalation is by oral inhalation.

33. The method of claim 30, wherein inhalation is by nasal inhalation.

34. The method of claim 30, wherein the composition is administered on as an as needed basis.

35. The method of claim 30, wherein the condition, disease or disorder is selected from the group consisting of asthma and chronic obstructive pulmonary disease.

36. A pulmonary drug delivery device, comprising: a pharmaceutical formulation comprised of a therapeutically effective amount of at least one bronchodilator, a therapeutically effective amount of at least one vasoconstrictor, and a therapeutically effective amount of at least one corticosteroid; and a means for housing and dispensing unit dosages of the formulation.

37. The device of claim 36, which further comprises a pharmaceutically acceptable carrier suitable for pulmonary drug administration.

38. The device of claim 36, comprising a dry powder inhaler, metered-dose inhaler, nebulizer or pump spray bottle.

39. The device of claim 36, in the form of a dry powder inhaler.

40. A dry powder inhaler for orienting and positioning a capsule containing a pharmaceutical formulation to be administered by inhalation, comprising:
a dispensing chamber containing a capsule of a dry powder pharmaceutical formulation comprised of a therapeutically effective amount of at least one bronchodilator, a therapeutically effective amount of at least one vasoconstrictor, and a therapeutically effective amount of at least one corticosteroid, and a pharmaceutically acceptable carrier suitable for pulmonary drug administration;
a tube for receiving the capsule to be oriented and dispensed;
a ramp surface extending substantially across the tube from one wall to an opposite wall thereof; and
an elongate dispensing passage having a diameter less than that of the tube and sized to receive the capsule only when the elongate axis of the capsule is generally parallel to the axis of the passage, the passage extending from an inlet end formed by an aperture in the ramp's surface to a dispensing outlet, the passage being adjacent to one wall of the tube such that the axis of the passage is parallel to, but radially offset from, an axis of the tube,
whereby when the inhaler is positioned with the passage below the tube and the axis of the passage is substantially vertical, a capsule located in the tube is guided by the ramp surface towards the inlet end of the passage.

41. A dosage form containing a pharmaceutical composition for pulmonary drug administration, the pharmaceutical composition comprising a therapeutically effective amount of at least one bronchodilator, a therapeutically effective amount of at least one vasoconstrictor, and a therapeutically effective amount of at least one corticosteroid.

42. The dosage form of claim 41, which further comprises a pharmaceutically acceptable carrier suitable for pulmonary drug administration.

43. The dosage form of claim 41, in the form of a capsule.

44. The dosage form of claim 43, wherein the capsule is a hydroxypropyl methylcellulose capsule.

45. The dosage form of claim 41, wherein the therapeutically effective amount of the bronchodilator is in the range of about 1 to 1500 μg.

46. The dosage form of claim 41, wherein the therapeutically effective amount of the vasoconstrictor is within the range of about 10 to 5000 μg.

47. The dosage form of claim 41, wherein the therapeutically effective amount of the corticosteroid is within the range of about 1 to 2000 μg.

48. The dosage form of claim 41, which further comprises a pharmaceutically acceptable carrier.

49. The dosage form of claim 48, wherein the carrier is selected from the group consisting of fructose, galactose, glucose, lactitol, lactose, maltitol, maltose, mannitol, melezitose, myoinositol, palatinite, raffinose, stachyose, sucrose, trehalose, xylitol, hydrates and combinations thereof.
